(19) United States
(12) Patent Application Publication
(10) Pub. No.: US 2016/0151265 A1
(43) Pub. Date: Jun. 2, 2016

(54) ANTIPERSPIRANTS WITH REDUCED ITCHING EFFECT

(71) Applicant: Henkel AG & Co. KGaA, Duesseldorf (DE)

(72) Inventors: Thomas Doering, Dormagen (DE); Natascha Schevardo, Erkrath (DE)

(21) Appl. No.: 15/016,383

(22) Filed: Feb. 5, 2016

Related U.S. Application Data
(63) Continuation of application No. PCT/DE2014/200326, filed on Jul. 17, 2014.

(30) Foreign Application Priority Data
Aug. 19, 2013 (DE) 10 2013 216 381.9

(51) Int. Cl. A61K 8/46; A61K 8/44; A61Q 15/00; A61K 8/24; A61K 8/86; A61K 8/26; A61K 8/49

(52) U.S. Cl. CPC A61K 8/466; A61K 8/26; A61K 8/44; A61K 8/4953; A61K 8/24; A61K 8/86; A61Q 15/00; A61K 2800/75

(57) ABSTRACT

A cosmetic or dermatological antiperspirant composition which includes, based on the weight of the composition: a) at least one antiperspirant active ingredient, b) 0-5 wt. % of taurine, c) 0-5 wt. % of glycine, d) 0-5 wt. % of ectoine, e) 0-2 wt. % ethylene diamine tetra acetic acid and/or the salts thereof, f) 0-2 wt. % of ethylenediaminedisuccinic acid and/or the salts thereof, g) 0-2 wt. % of citric acid and/or the salts thereof, h) 0-2 wt. % of gluconic acid and/or the salts thereof, i) 0-2 wt. % of zeolite A, j) 0-2 wt. % of sodium tripolyphosphate, and k) 0-2 wt. % of sodium hexametaphosphate, wherein the total quantity of the contents b), c), and d) equals 0.1-5 wt. %, and the total quantity of the contents e), f), g), h), i), j), and k) equals 0.01-2 wt. %.

### FIELD OF THE INVENTION

The present invention generally relates to cosmetic and dermatological antiperspirant compositions having a reduced irritation potential.

### BACKGROUND OF THE INVENTION

Commercially available perspiration-inhibiting compositions, hereinafter also referred to as antiperspirants, include at least one water-soluble astringent inorganic and organic salt of aluminum, zinc or selected mixed aluminum-zirconium salts as the active antiperspirant ingredient.

The active antiperspirant ingredients have no direct influence on the activity of the sweat glands, but instead minimize secretion of sweat by constricting the discharge ducts. The Al salts bring about a perspiration-inhibiting action on the treated areas of skin by clogging the superficial areas of the sweat gland ducts as a result of Al mucopolysaccharide precipitates. Antiperspirant compositions are usually applied and used in the axilla region. Due to its location and function, the axillary skin has a weak barrier action. The skin in the axillary area is thus more sensitive than the rest of the skin on the body, frequently at least just as sensitive as the skin of the face. Many consumers consider hairless axillary skin to be more attractive and more conducive to hygiene, since hair growth enlarges the sweat-bearing surface area in the axilla region and thus contributes to increased development of body odor. As a result, regular shaving of the axillary hair is part of many consumers' hygiene routine. Shaving the armpits irritates the skin mechanically, and additionally weakens the barrier function. After shaving, an antiperspirant and/or deodorant cosmetic preparation, for example a deodorant spray or stick, is usually applied onto the axillary skin. The active antiperspirant ingredients, for example due to the acidic pH value of the same, generally have considerable irritation potential which, on the—mechanically irritated—axillary skin can cause reddened skin, unpleasant stinging, feeling of tightness and/or itching.

As a result, there is a constant need for formulations that are particularly gentle on the skin and suitable as carriers for cosmetic and dermatological antiperspirant and deodorant compositions. A need furthermore exists for formulations that are particularly gentle on the skin and, as carriers for cosmetic and dermatological antiperspirant and deodorant compositions, may be capable of making the active antiperspirant and deodorant ingredients, which occasionally have an irritating effect on the skin when used regularly and/or on mechanically or chemically irritated skin, more gentle on the skin or of relieving the irritating effect of the same.

The use of taurine for reducing the itching effect of deodorant or antiperspirant compositions is known from DE 10 2006 062 433 A1. For sensitive individuals, however, the compositions disclosed therein have been found not to be entirely itch-free.

It was the object of the present invention to further minimize the itching effect caused by active antiperspirant ingredients and to provide products that can also be used successfully by sensitive consumers. It was a further object of the present invention to find active ingredients and/or carriers for cosmetic and dermatological antiperspirant and deodorant compositions which reduce the stinging and/or itching of the skin during application yet again.

Surprisingly, it was found that taurine and/or glycine and/or ectoine, each in combination with a particular complexing agent, achieve the described objects.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A cosmetic or dermatological antiperspirant composition, comprising, based on the weight thereof, at least one active antiperspirant ingredient, 0 to 5 wt. % taurine, 0 to 5 wt. % glycine, 0 to 5 wt. % ectoine, 0 to 2 wt. % ethylenediaminetetraacetic acid and/or the salts thereof, 0 to 2 wt. % ethylenediaminedisuccinic acid and/or the salts thereof, 0 to 2 wt. % citric acid and/or the salts thereof, 0 to 2 wt. % gluconic acid and/or the salts thereof, 0 to 2 wt. % zeolite A, 0 to 2 wt. % sodium tripolyphosphate, 0 to 2 wt. % sodium hexametaphosphate, with the proviso that the total amount of ingredients b), c) and d) is 0.1 to 5 wt. %, and the total amount of ingredients e), f), g), h), i), j) and k) is 0.01 to 2 wt. %.

Use of at least one active ingredient, selected from: taurine, glycine, or ectoine, in combination with at least one active ingredient, selected from: ethylenediaminetetraacetate, ethylenediaminetetraacetic acid and/or the salts thereof, ethylenediaminedisuccinic acid and/or the salts thereof, citric acid and/or the salts thereof, gluconic acid and/or the salts thereof, zeolite A, sodium tripolyphosphate, or sodium hexametaphosphate, in a cosmetically or dermatologically tolerable carrier, comprising at least one active antiperspirant ingredient, to improve the skin tolerance and/or the nourishing action of the composition and/or to reduce or prevent stinging of the skin and/or itching caused by the contact of the skin with the composition.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

In a first embodiment, the invention relates to a cosmetic or dermatological antiperspirant composition, including, based on the weight thereof,
a) at least one active antiperspirant ingredient,
b) 0 to 5 wt. % taurine,
c) 0 to 5 wt. % glycine,
d) 0 to 5 wt. % ectoine,
e) 0 to 2 wt. % ethylenediaminetetraacetic acid and/or the salts thereof,
f) 0 to 2 wt. % ethylenediaminedisuccinic acid and/or the salts thereof,
g) 0 to 2 wt. % citric acid and/or the salts thereof,
h) 0 to 2 wt. % gluconic acid and/or the salts thereof,
i) 0 to 2 wt. % zeolite A,
j) 0 to 2 wt. % sodium tripolyphosphate,
k) 0 to 2 wt. % sodium hexametaphosphate,
   with the proviso that
the total amount of ingredients b), c) and d) is 0.1 to 5 wt. %, and
the total amount of ingredients e), f), g), h), i), j) and k) is 0.01 to 2 wt. %.

The information in % by weight refers in each case to the total weight of the agent according to the invention, unless indicated otherwise. The terms “agent according to the invention” and “composition according to the invention” are used as synonyms in the present application. “Normal conditions” within the meaning of the present application refer to a temperature of 20° C. and a pressure of 1013 mbar. Melting point information likewise refers to a pressure of 1013 mbar. The compositions according to the invention include at least one active antiperspirant ingredient, preferably 3 to 25 wt. %, preferably 5 to 22 wt. %, and in particular 10 to 20 wt. % of at least one activated antiperspirant aluminum or aluminum-zirconium salt.

Particularly preferred agents according to the invention include at least one antiperspirant aluminum salt in a total amount of 2 to 40 wt. %, preferably 8 to 35 wt. %, particularly preferably 10 to 28 wt. %, and exceptionally preferably 12 to 20 wt. %, wherein the information in % by weight is based on the total weight of the constitutional water-free and ligand-free active substance (USP) in the composition.

It may be preferred according to the invention that the cosmetic agent is free of zirconium compounds.

The antiperspirant aluminum salts are preferably selected from the water-soluble astringent inorganic and organic salts of aluminum and aluminum-zirconium mixtures. According to the invention, aluminosilicates and zeolites are not covered by the active antiperspirant ingredients. According to the invention, water solubility shall be understood to mean a solubility of at least 3 wt. % at 20° C., which is to say that amounts of at least 3 g of the active antiperspirant ingredient are soluble in 97 g water at 20° C.

Particularly preferred active antiperspirant ingredients are selected from aluminum chlorohydrate, in particular aluminum chlorohydrate of general formula [Al₂(OH)₅CI.1-6H₂O]ₙ, preferably [Al₂(OH)₅CI.2-3H₂O]ₙ, which may be present in non-activated or in activated (depolymerized) form, and aluminum chlorohydrate of general formula [Al₂(OH)₄Cl₂.1-6H₂O]ₙ, preferably [Al₂(OH)₄Cl₂.2-3H₂O]ₙ, which may be present in non-activated or in activated (depolymerized) form.

Furthermore preferred are aluminum sesquichlorohydrate, aluminum dichlorohydrate, aluminum chlorohydrex propylene glycol (PG) or aluminum chlorohydrex polyethylene glycol (PEG), aluminum or aluminum-zirconium glycol complexes, such as aluminum or aluminum-zirconium propylene glycol complexes, aluminum sesquichlorohydrex PG or aluminum sesquichlorohydrex PEG, aluminum dichlorohydrex PG or aluminum dichlorohydrex PEG, aluminum hydroxide, furthermore selected from the aluminum-zirconium chlorohydrates, such as aluminum-zirconium trichlorohydrate, aluminum-zirconium tetrachlorohydrate, aluminum-zirconium pentachlorohydrate, aluminum-zirconium octachlorohydrate, the aluminum-zirconium chlorohydrate glycine complexes, such as aluminum-zirconium trichlorohydrex glycine, aluminum-zirconium tetrachlorohydrex glycine, aluminum-zirconium pentachlorohydrex glycine, aluminum-zirconium octachlorohydrex glycine, potassium aluminum sulfate (KAI(SO₄)₂.12H₂O, alum), aluminum undecylenoyl collagen amino acid, sodium aluminum lactate+aluminum sulfate, sodium aluminum chlorohydroxy lactate, aluminum bromohydrate, aluminum chloride, the aluminum salts of lipoamino acids, aluminum sulfate, aluminum lactate, aluminum chlorhydroxy allantoinate and sodium aluminum chlorhydroxy lactate.

Particularly preferred active antiperspirant ingredients according to the invention are selected from what are known as “activated” aluminum and aluminum-zirconium salts, which are also referred to as enhanced-activity active antiperspirant ingredients. Such active ingredients are known from the prior art and are also commercially available. Activated aluminum and aluminum-zirconium salts are generally produced by heat treating a relatively dilute solution of the salt (such as approximately 10 wt. % salt), so as to increase the HPLC peak 4 to peak 3 area ratio of the same. The activated salt can subsequently be dried to obtain a powder, in particular spray-dried. In addition to spray drying, drum drying also suited, for example.

Activated aluminum and aluminum-zirconium salts typically have an HPLC peak 4 to peak 3 area ratio of at least 0.4, preferably at least 0.7, particularly preferably at least 0.9, wherein at least 70% of the aluminum can be assigned to these peaks.

Activated aluminum and aluminum-zirconium salts do not necessarily have to be used in the form of spray-dried powder. Likewise preferred active antiperspirant ingredients according to the invention are non-aqueous solutions or solubilizates of an activated antiperspirant aluminum or aluminum-zirconium salt, which, as a result of the addition of an effective amount of a polyhydric alcohol comprising 3 to 6 carbon atoms and 3 to 6 hydroxyl groups, preferably propylene glycol, sorbitol and pentaerythritol, are stabilized against the loss of the activation against the rapid decrease in the HPLC peak 4:peak 3 area ratio of the salt. For example, preferred are compositions that comprise, in percent by weight (USP): 18 to 45 wt. % of an activated aluminum or aluminum-zirconium salt, 55 to 82 wt. % of at least one anhydrous polyhydric alcohol comprising 3 to 6 carbon atoms and 3 to 6 hydroxyl groups, preferably 1,2-propylene glycol, 1,3-butylene glycol, diethylene glycol, dipropylene glycol, glycerol, sorbitol and pentaerythritol, particularly preferably 1,2-propylene glycol.

Particularly preferred are also complexes of activated antiperspirant aluminum or aluminum-zirconium salts, comprising a polyhydric alcohol, which include 20 to 50 wt. %, particularly preferably 20 to 42 wt. %, activated antiperspirant aluminum or aluminum-zirconium salt and 2 to 16 wt. % molecularly bound water, wherein the remainder to make up to 100 wt. % is at least one polyhydric alcohol comprising 3 to 6 carbon atoms and 3 to 6 hydroxyl groups. Propylene glycol, propylene glycol/sorbitol mixtures, and propylene glycol/pentaerythritol mixtures are preferred such alcohols.

Further preferred active antiperspirant ingredients are alkaline calcium aluminum salts. These salts are produced by reacting calcium carbonate with aluminum chlorhydroxide or aluminum chloride and aluminum powder, or by adding calcium chloride dihydrate to aluminum chlorhydroxide.

Further preferred active antiperspirant ingredients are aluminum-zirconium complexes buffered with salts of amino acids, in particular with alkali and alkaline earth glycinates.

Further preferred active antiperspirant ingredients are activated aluminum or aluminum-zirconium salts, comprising 5 to 78 wt. % (USP) of an activated antiperspirant aluminum or aluminum-zirconium salt, an amino acid or hydroxyalkanoic acid in such an amount so as to provide an (amino acid or hydroxyalkanoic acid) to (Al+Zr) weight ratio of 2:1 to 1:20, and preferably 1:1 to 1:10, and a water-soluble calcium salt in such an amount so as to provide a Ca:(Al+Zr) weight ratio of 1:1 to 1:28, and preferably 1:2 to 1:25. Particularly preferred solid activated antiperspirant salt compositions include 48 to 78 wt. % (USP), preferably 66 to 75 wt. %, of an activated aluminum or aluminum-zirconium salt and 1 to 16 wt. %, preferably 4 to 13 wt. %, molecularly bound water (hydration water), furthermore an amount of water-soluble calcium salt that is such that the Ca:(Al+Zr) weight ratio is 1:1 to 1:28, preferably 1:2 to 1:25, and an amount of amino acid that is such that the amino acid to (Al+Zr) weight ratio is 2:1 to 1:20, preferably 1:1 to 1:10.

Further particularly preferred solid antiperspirant activated salt compositions include 48 to 78 wt. % (USP), preferably 66 to 75 wt. %, of an activated aluminum or aluminum-zirconium salt and 1 to 16 wt. %, preferably 4 to 13 wt. %, molecularly bound water (hydration water), furthermore an amount of water-soluble calcium salt that is such that the Ca:(Al+Zr) weight ratio is 1:1 to 1:28, preferably 1:2 to 1:25, and an amount of glycine that is such that the glycine to (Al+Zr) weight ratio is 2:1 to 1:20, preferably 1:1 to 1:10.

Further particularly preferred solid antiperspirant activated salt compositions include 48 to 78 wt. % (USP), preferably 66 to 75 wt. %, of an activated aluminum or aluminum-zirconium salt and 1 to 16 wt. %, preferably 4 to 13 wt. %, molecularly bound water, furthermore an amount of water-soluble calcium salt that is such that the Ca:(Al+Zr) weight ratio is 1:1 to 1:28, preferably 1:2 to 1:25, and an amount of hydroxyalkanoic acid that is such that the hydroxyalkanoic acid to (Al+Zr) weight ratio is 2:1 to 1:20, preferably 1:1 to 1:10.

Amino acids that are preferred for the stabilization of the antiperspirant salts are selected from the group consisting of glycine, alanine, leucine, isoleucine, β-alanine, valine, cysteine, serine, tryptophan, phenylalanine, methionine, β-amino-n-butyric acid and γ-amino-n-butyric acid, and the salts thereof, each in the D form, the L form, and the DL form, glycine being particularly preferred.

Hydroxyalkanoic acids that are preferred for the stabilization of the antiperspirant salts are selected from glycolic acid and lactic acid.

Further preferred activated aluminum salts are those of general formula Al₂(OH)₆₋ₐXa, where X is Cl, Br, I or NO₃, and “a” is a value from 0.3 to 5, preferably from 0.8 to 2.5, and particularly preferably 1 to 2, so that the molar ratio of Al:X is 0.9:1 to 2.1:1. In general, a small amount of hydration water is associatively bound in these salts, typically 1 to 6 moles of water per mole of salt. Aluminum chlorohydrate is particularly preferred (which is to say X is Cl in the aforementioned formula), and specifically 5/6 basic aluminum chlorohydrate, where “a” is 1, so that the molar ratio of aluminum to chlorine is 1.9:1 to 2.1:1.

Preferred activated aluminum-zirconium salts include those that represent mixtures or complexes of the above-described aluminum salts with zirconium salts of formula ZrO(OH)_{2-pb}Y_{b}, where Y is Cl, Br, I, NO₃ or SO₄, b is a rational number from 0.8 to 2, and p is the valence of Y, as they are disclosed in U.S. Pat. No. 6,074,632, for example. In general, a small amount of hydration water is likewise associatively bound in the zirconium salts, typically 1 to 7 moles of water per mole of salt. The zirconium salt is preferably zirconyl hydroxychloride of formula ZrO(OH)_{2-b}Cl_{b}, where b is a rational number from 0.8 to 2, preferably 1.0 to 1.9. Preferred aluminum-zirconium salts have an Al:Zr molar ratio of 2 to 10 and a metal:(X+Y) ratio of 0.73 to 2.1, preferably 0.9 to 1.5. A particularly preferred salt is aluminum-zirconium chlorohydrate (which is to say X and Y are CI), which has an Al:Zr ratio of 2 to 10 and a metal:Cl molar ratio of 0.9 to 2.1. The term aluminum-zirconium chlorohydrate encompasses the tri-, tetra-, penta- and octachlorohydrate forms.

Preferred antiperspirant aluminum-zirconium salts have a metal-to-chloride molar ratio of 0.9 to 1.5, preferably 0.9 to 1.3, particularly preferably 0.9 to 1.1.

Zirconium-free aluminum salts that are particularly preferred according to the invention have a molar metal-to-chloride ratio of 1.9 to 2.1. Zirconium-free aluminum sesquichlorohydrates that are particularly preferred according to the invention have a metal-to-chloride molar ratio of 1.5:1 to 1.8:1. Preferred aluminum-zirconium chlorohydrates in general have the empirical formula AlₙZr(OH)_{[3n+4-m(n+1)]}(Cl)_{[m(n+1)]} where n=2.0 to 10.0, preferably 3.0 to 8.0, m=0.77 to 1.11 (corresponding to a metal (Al+Zr)-to-chloride molar ratio of 1.3 to 0.9), preferably m=0.91 to 1.11 (corresponding to M:Cl=1.1 to 0.9), and particularly preferably m=1.00 to 1.11 (corresponding to M:Cl=1.0 to 0.9), further very preferably m=1.02 to 1.11 (corresponding to M:Cl=0.98 to 0.9) and very preferably m=1.04 to 1.11 (corresponding to M:Cl=0.96 to 0.9). In general, a small amount of hydration water is associatively bound in these salts, typically 1 to 6 moles of water per mole of salt, corresponding to 1 to 16 wt. %, preferably 4 to 13 wt. %, hydration water.

The preferred aluminum-zirconium chlorohydrates are usually associated with an amino acid to prevent polymerization of the zirconium species during production. Preferred stabilizing amino acids are selected from the group consisting of glycine, alanine, leucine, isoleucine, β-alanine, cysteine, valine serine, tryptophan, phenylalanine, methionine, β-amino-n-butyric acid and γ-amino-n-butyric acid, and the salts thereof, each in the D form, the L form, and the DL form, glycine being particularly preferred. The amino acid is present in the salt in an amount of 1 to 3 moles, preferably 1.3 to 1.8 moles, in each case per mole of zirconium.

Preferred antiperspirant salts are aluminum-zirconium tetrachlorohydrates (Al:Zr=2 to 6; M:Cl=0.9 to 1.3), in particular salts having a metal-to-chloride molar ratio of 0.9 to 1.1, preferably 0.9 to 1.0.

Aluminum-zirconium chlorohydrate glycine salts that are stabilized with betaine ((CH₃)₃N⁺—CH₂—COO⁻) are furthermore preferred according to the invention. Particularly preferred corresponding compounds have a total molar (betaine+glycine)/Zr ratio of (0.1 to 3.0):1, preferably (0.7 to 1.5):1 and a molar ratio of betaine to glycine of at least 0.001:1.

In a particularly preferred embodiment according to the invention, what is known as an “activated” salt is present as a particularly effective antiperspirant salt, in particular one having a high HPLC peak 5 aluminum content, in particular having a peak 5 area of at least 33%, particularly preferably at least 45%, based on the total surface area under the peaks 2-5, as measured with HPLC, of a 10% by weight aqueous solution of the active ingredient under conditions in which the aluminum species are dissolved in at least four successive peaks (referred to as peaks 2-5). Preferred aluminum-zirconium salts are those having a high HPLC peak 5 aluminum content (also referred to as “E⁵AZCH”).

Furthermore, preferred activated “E⁵AZCH” salts are those having an HPLC peak 4 to peak 3 area ratio of at least 0.4, preferably at least 0.7, particularly preferably at least 0.9. Further particularly preferred active antiperspirant ingredients are such aluminum-zirconium salts having a high HPLC peak 5 aluminum content which are additionally stabilized with a water-soluble strontium salt and/or with a water-soluble calcium salt.

The formulation of the agents according to the invention in a particular form of administration, such as a roll-on antiperspirant or an antiperspirant stick or antiperspirant gel, is preferably dependent on the requirements of the intended purpose.

Agents according to the invention can be present in solid, semi-solid, liquid, disperse, emulsified, suspended or gel-like form.

In a particularly preferred embodiment, the agents according to the invention are present in liquid or viscous, flowable form. The application can preferably take place by way of a roller ball applicator. Such rollers comprise a ball that is mounted in a ball bed and can be moved by motion across a surface. The ball takes up a small amount of the agent to be distributed in this process and delivers the same to the surface to be treated. The packaging for the agents according to the invention can be opaque, but may also be transparent or translucent.

An additional ingredient, or additional ingredients, that the agents according to the invention include are 0 to 5 wt. % taurine, 0 to 5 wt. % glycine, 0 to 5 wt. % ectoine, with the proviso that the total amount of these three ingredients b), c) and d) is 0.1 to 5 wt. %.

Preferred agents according to the invention comprise 0.1 to 5 wt. %, preferably 0.2 to 4 wt. %, more preferably 0.3 to 3 wt. %, particularly preferably 0.4 to 2 wt. %, and in particular 0.5 to 1.5 wt. % taurine.

If taurine is used in an amount of less than 5 wt. %, the agents according to the invention may also include ectoine, in addition to taurine. Ectoine can also be used alone, of course, wherein, regardless of the presence of taurine, preferred compositions according to the invention comprise 0.1 to 5 wt. %, preferably 0.2 to 4 wt. %, more preferably 0.3 to 3 wt. %, particularly preferably 0.4 to 2 wt. %, and in particular 0.5 to 1.5 wt. % ectoine.

If the total amount of taurine and ectoine is less than 5 wt. %, the agents according to the invention may also include glycine, in addition to taurine and ectoine. Glycine can also be used only in combination with taurine, or only in combination with ectoine, or alone, of course, wherein, regardless of the presence of taurine and/or ectoine, preferred compositions according to the invention comprise 0.1 to 5 wt. %, preferably 0.2 to 4 wt. %, more preferably 0.3 to 3 wt. %, particularly preferably 0.4 to 2 wt. %, and in particular 0.5 to 1.5 wt. % glycine.

Particularly preferred compositions according to the invention comprise 0.1 to 5 wt. %, preferably 0.2 to 4 wt. %, more preferably 0.3 to 3 wt. %, particularly preferably 0.4 to 2 wt. %, and in particular 0.5 to 1.5 wt. % taurine, and 0.1 to 5 wt. %, preferably 0.2 to 4 wt. %, more preferably 0.3 to 3 wt. %, particularly preferably 0.4 to 2 wt. %, and in particular 0.5 to 1.5 wt. % ectoine.

Further preferred compositions according to the invention comprise 0.1 to 5 wt. %, preferably 0.2 to 4 wt. %, more preferably 0.3 to 3 wt. %, particularly preferably 0.4 to 2 wt. %, and in particular 0.5 to 1.5 wt. % taurine, and 0.1 to 5 wt. %, preferably 0.2 to 4 wt. %, more preferably 0.3 to 3 wt. %, particularly preferably 0.4 to 2 wt. %, and in particular 0.5 to 1.5 wt. % glycine.

Likewise preferred compositions according to the invention comprise 0.1 to 5 wt. %, preferably 0.2 to 4 wt. %, more preferably 0.3 to 3 wt. %, particularly preferably 0.4 to 2 wt. %, and in particular 0.5 to 1.5 wt. % glycine, and 0.1 to 5 wt. %, preferably 0.2 to 4 wt. %, more preferably 0.3 to 3 wt. %, particularly preferably 0.4 to 2 wt. %, and in particular 0.5 to 1.5 wt. % ectoine.

Preferred compositions according to the invention in particular comprise 0.1 to 5 wt. %, preferably 0.2 to 4 wt. %, more preferably 0.3 to 3 wt. %, particularly preferably 0.4 to 2 wt. %, and in particular 0.5 to 1.5 wt. % taurine, and 0.1 to 5 wt. %, preferably 0.2 to 4 wt. %, more preferably 0.3 to 3 wt. %, particularly preferably 0.4 to 2 wt. %, and in particular 0.5 to 1.5 wt. % ectoine, and 0.1 to 5 wt. %, preferably 0.2 to 4 wt. %, more preferably 0.3 to 3 wt. %, particularly preferably 0.4 to 2 wt. %, and in particular 0.5 to 1.5 wt. % glycine. An additional ingredient, or additional ingredients, that the agents according to the invention include are 0 to 2 wt. % ethylenediaminetetraacetic acid and/or the salts thereof, 0 to 2 wt. % ethylenediaminedisuccinic acid and/or the salts thereof, 0 to 2 wt. % citric acid and/or the salts thereof, 0 to 2 wt. % gluconic acid and/or the salts thereof, 0 to 2 wt. % zeolite A, 0 to 2 wt. % sodium tripolyphosphate, 0 to 2 wt. % sodium hexametaphosphate, with the proviso that the total amount of these three ingredients e), f), g), h), i), j) and k) is 0.01 to 2 wt. %.

Preferred agents according to the invention comprise 0.01 to 2 wt. %, preferably 0.02 to 1 wt. %, more preferably 0.03 to 0.5 wt. %, particularly preferably 0.04 to 0.25 wt. %, and in particular 0.05 to 0.2 wt. % ethylenediaminetetraacetic acid and/or the salts thereof.

In addition to the free acid, in particular the salts thereof, disodium ethylenediaminetetraacetate (Na₂H₂EDTA, sodium edetate), tetrasodium ethylenediaminetetraacetate (Na₄EDTA), and calcium disodium ethylenediaminetetraacetate (CaNa₂EDTA, E385) can be used according to the invention. The combinations of taurine and ethylenediaminetetraacetic acid and/or the salts thereof have been found to be particularly suitable according to the invention since here itch alleviation is the most effective. Preferred agents according to the invention thus comprise 0.1 to 5 wt. %, preferably 0.2 to 4 wt. %, more preferably 0.3 to 3 wt. %, particularly preferably 0.4 to 2 wt. %, and in particular 0.5 to 1.5 wt. % taurine, and 0.01 to 2 wt. %, preferably 0.02 to 1 wt. %, more preferably 0.03 to 0.5 wt. %, particularly preferably 0.04 to 0.25 wt. %, and in particular 0.05 to 0.2 wt. % ethylenediaminetetraacetic acid.

Further preferred agents according to the invention thus comprise 0.1 to 5 wt. %, preferably 0.2 to 4 wt. %, more preferably 0.3 to 3 wt. %, particularly preferably 0.4 to 2 wt. %, and in particular 0.5 to 1.5 wt. % taurine, and 0.01 to 2 wt. %, preferably 0.02 to 1 wt. %, more preferably 0.03 to 0.5 wt. %, particularly preferably 0.04 to 0.25 wt. %, and in particular 0.05 to 0.2 wt. % disodium ethylenediaminetetraacetic acid (Na₂H₂EDTA).

Further preferred agents according to the invention thus comprise 0.1 to 5 wt. %, preferably 0.2 to 4 wt. %, more preferably 0.3 to 3 wt. %, particularly preferably 0.4 to 2 wt. %, and in particular 0.5 to 1.5 wt. % taurine, and 0.01 to 2 wt. %, preferably 0.02 to 1 wt. %, more preferably 0.03 to 0.5 wt. %, particularly preferably 0.04 to 0.25 wt. %, and in particular 0.05 to 0.2 wt. % tetrasodium ethylenediaminetetraacetic acid (Na₄EDTA).

Further preferred agents according to the invention thus comprise 0.1 to 5 wt. %, preferably 0.2 to 4 wt. %, more preferably 0.3 to 3 wt. %, particularly preferably 0.4 to 2 wt. %, and in particular 0.5 to 1.5 wt. % taurine, and 0.01 to 2 wt. %, preferably 0.02 to 1 wt. %, more preferably 0.03 to 0.5 wt. %, particularly preferably 0.04 to 0.25 wt. %, and in particular 0.05 to 0.2 wt. % calcium disodium ethylenediaminetetraacetic acid (CaNa₂EDTA).

Preferably an excess of taurine relative to the complexing agent is used in these preferred embodiments of the present invention. Preferred compositions according to the invention are characterized by comprising 0.1 to 5 wt. %, preferably 0.2 to 4 wt. %, more preferably 0.3 to 3 wt. %, particularly preferably 0.4 to 2 wt. %, and in particular 0.5 to 1.5 wt. % taurine, and 0.01 to 2 wt. %, preferably 0.02 to 1 wt. %, more preferably 0.03 to 0.5 wt. %, particularly preferably 0.04 to 0.25 wt. %, and in particular 0.05 to 0.2 wt. % ethylenediaminetetraacetic acid and/or the salts thereof, wherein the weight ratio of taurine to ethylenediaminetetraacetic acid and/or the salts thereof is 2:1 to 100:1, preferably 5:1 to 50:1, more preferably 7:1 to 25:1, and in particular 9:1 to 12:1.

Likewise preferred agents according to the invention comprise 0.01 to 2 wt. %, preferably 0.02 to 1 wt. %, more preferably 0.03 to 0.5 wt. %, particularly preferably 0.04 to 0.25 wt. %, and in particular 0.05 to 0.2 wt. % ethylenediaminedisuccinic acid and/or the salts thereof.

In addition to a very good itch-alleviating action in combination with taurine, ectoine, glycine, the agents comprising EDDS have the advantage of improved biodegradability of the complexing agent.

In addition to the free acid, in particular the salts thereof, disodium ethylenediamine disuccinate (Na₂H₂EDDS) and trisodium ethylenediamine disuccinate (Na₃HEDDS), can be used according to the invention. The combinations of taurine and EDDS and/or the salts thereof have been found to be particularly suitable according to the invention since here itch alleviation is the most effective.

Preferred agents according to the invention thus comprise 0.1 to 5 wt. %, preferably 0.2 to 4 wt. %, more preferably 0.3 to 3 wt. %, particularly preferably 0.4 to 2 wt. %, and in particular 0.5 to 1.5 wt. % taurine, and 0.01 to 2 wt. %, preferably 0.02 to 1 wt. %, more preferably 0.03 to 0.5 wt. %, particularly preferably 0.04 to 0.25 wt. %, and in particular 0.05 to 0.2 wt. % ethylenediaminedisuccinic acid (EDDS).

Further preferred agents according to the invention thus comprise 0.1 to 5 wt. %, preferably 0.2 to 4 wt. %, more preferably 0.3 to 3 wt. %, particularly preferably 0.4 to 2 wt. %, and in particular 0.5 to 1.5 wt. % taurine, and 0.01 to 2 wt. %, preferably 0.02 to 1 wt. %, more preferably 0.03 to 0.5 wt. %, particularly preferably 0.04 to 0.25 wt. %, and in particular 0.05 to 0.2 wt. % disodium ethylenediamine disuccinate (Na₂H₂EDDS).

Further preferred agents according to the invention thus comprise 0.1 to 5 wt. %, preferably 0.2 to 4 wt. %, more preferably 0.3 to 3 wt. %, particularly preferably 0.4 to 2 wt. %, and in particular 0.5 to 1.5 wt. % taurine, and 0.01 to 2 wt. %, preferably 0.02 to 1 wt. %, more preferably 0.03 to 0.5 wt. %, particularly preferably 0.04 to 0.25 wt. %, and in particular 0.05 to 0.2 wt. % trisodium ethylenediamine disuccinate (Na₃HEDDS).

Instead of or in addition to EDTA or EDDS, or the salts thereof, it is also possible to use citric acid and/or the salts thereof, gluconic acid and/or the salts thereof, zeolite A, sodium tripolyphosphate, sodium hexametaphosphate.

Sodium phosphates, in particular together with ectoine, exhibit particularly pronounced effects, while zeolite A together with taurine and ectoine is most effective.

Further preferred agents according to the invention thus comprise 0.1 to 5 wt. %, preferably 0.2 to 4 wt. %, more preferably 0.3 to 3 wt. %, particularly preferably 0.4 to 2 wt. %, and in particular 0.5 to 1.5 wt. % ectoine, and 0.01 to 2 wt. %, preferably 0.02 to 1 wt. %, more preferably 0.03 to 0.5 wt. %, particularly preferably 0.04 to 0.25 wt. %, and in particular 0.05 to 0.2 wt. % sodium tripolyphosphate.

Further preferred agents according to the invention thus comprise 0.1 to 5 wt. %, preferably 0.2 to 4 wt. %, more preferably 0.3 to 3 wt. %, particularly preferably 0.4 to 2 wt. %, and in particular 0.5 to 1.5 wt. % ectoine, and 0.01 to 2 wt. %, preferably 0.02 to 1 wt. %, more preferably 0.03 to 0.5 wt. %, particularly preferably 0.04 to 0.25 wt. %, and in particular 0.05 to 0.2 wt. % sodium hexametaphosphate.

Further preferred agents according to the invention thus comprise 0.1 to 5 wt. %, preferably 0.2 to 4 wt. %, more preferably 0.3 to 3 wt. %, particularly preferably 0.4 to 2 wt. %, and in particular 0.5 to 1.5 wt. % taurine, and 0.01 to 2 wt. %, preferably 0.02 to 1 wt. %, more preferably 0.03 to 0.5 wt. %, particularly preferably 0.04 to 0.25 wt. %, and in particular 0.05 to 0.2 wt. % zeolite A.

Further preferred agents according to the invention thus comprise 0.1 to 5 wt. %, preferably 0.2 to 4 wt. %, more preferably 0.3 to 3 wt. %, particularly preferably 0.4 to 2 wt. %, and in particular 0.5 to 1.5 wt. % ectoine, and 0.01 to 2 wt. %, preferably 0.02 to 1 wt. %, more preferably 0.03 to 0.5 wt. %, particularly preferably 0.04 to 0.25 wt. %, and in particular 0.05 to 0.2 wt. % zeolite A.

The compositions according to the invention preferably comprise 10 to 90 wt. %, particularly preferably 50 to 85 wt. %, exceptionally preferably 60 to 80 wt. %, more exceptionally preferably 65 to 75 wt. % water, in each case based on the total weight of the composition. “Water” within the meaning of the present application shall be understood to mean “free water”, which is to say water that is not present in the antiperspirant composition in the form of constitutional water, hydration water or similarly molecularly bound water. The content of constitutional water, hydration water or similarly molecularly bound water that is present in the components used, in particular in the active antiperspirant ingredients, does not constitute free water within the meaning of the present application. Free water is water which is present, for example, as a solvent or as a solvent component of other active ingredients in the composition according to the invention.

Particularly preferred compositions according to the invention comprise 10 to 80 wt. %, preferably 20 to 79 wt. %, more preferably 30 to 78 wt. %, particularly preferably 40 to 77 wt. %, and in particular 50 to 75 wt. % water.

Preferred compositions according to the invention comprise at least one oil-in-water emulsifier having an HLB value greater than 7 to 20, which is particularly preferably selected from non-ionic oil-in-water emulsifiers having an HLB value of greater than 7 to 20.

For ethoxylated products, the HLB value is calculated according to formula HLB=(100−L):5, where L is the weight proportion of the lipophilic groups, which is to say of the fatty alkyl or fatty acyl groups, in the ethylene oxide adducts, expressed in percent by weight.

Further preferred antiperspirant compositions according to the invention are characterized by comprising at least one non-ionic emulsifier having an HLB value in the range of 12 to 18. Preferred antiperspirant compositions according to the invention are characterized in that the non-ionic oil-in-water emulsifiers having an HLB value of greater than 7 to 20 are selected from the ethoxylated C₈ to C₂₄ alkanols comprising, on average, 10 to 100 moles ethylene oxide per mole, ethoxylated C₈ to C₂₄ carboxylic acids comprising, on average, 10 to 100 moles ethylene oxide per mole, comprising, on average, 20 to 100 moles ethylene oxide per mole of ethoxylated sorbitan monoesters of linear saturated and unsaturated C₁₂ to C₃₀ carboxylic acids, which may be hydroxylated, in particular those of myristic acid, palmitic acid, stearic acid, or of mixtures of these fatty acids, silicone copolyols comprising ethylene oxide units or ethylene oxide and propylene oxide units, alkyl monoglycosides and alkyl oligoglycosides comprising 8 to 22 carbon atoms in the alkyl group and the ethoxylated analogs thereof, ethoxylated sterols, partial esters of polyglycerols comprising n=2 to 10 glycerol units and comprising 1 to 4 saturated or unsaturated, linear or branched, optionally hydroxylated C₈ to C₃₀ fatty acid esters, provided that they have an HLB value of greater than 7 to 20, and mixtures of the aforementioned substances.

The ethoxylated C₈ to C₂₄ alkanols have the formula R¹O(CH₂CH₂O)ₙH, where R¹ denotes a linear or branched alkyl group and/or alkenyl group having 8 to 24 carbon atoms, and n, which represents the average number of ethylene oxide units per molecule, denotes numbers from 10 to 100, preferably 10 to 30 moles ethylene oxide on 1 mole capryl alcohol, 2-ethylhexanol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, and brassidyl alcohol, and the technical mixtures thereof. Adducts of 10 to 100 moles ethylene oxide on technical fatty alcohols comprising 12 to 18 carbon atoms, such as coconut, palm, palm kernel or tallow fatty alcohol, are also suitable.

The ethoxylated C₈ to C₂₄ carboxylic acids have the formula R¹O(CH₂CH₂O)ₙH, where R¹O denotes a linear or branched. saturated or unsaturated acyl group having 8 to 24 carbon atoms, and n, which represents the average number of ethylene oxide units per molecule, denotes numbers from 10 to 100, preferably 10 to 30 moles ethylene oxide on 1 mole caprylic acid, 2-ethyl-hexanoic acid, capric acid, lauric acid, isotridecanoic acid, myristic acid, cetylic acid, palmitoleic acid, stearic acid, isostearic acid, oleic acid, elaidic acid, petroselinic acid, arachyinic acid, gadoleic acid, behenic acid, erucic acid, and brassidic acid, and the technical mixtures thereof. Adducts of 10 to 100 moles ethylene oxide to technical fatty acids comprising 12 to 18 carbon atoms, such as coconut, palmitic, palm kernel or tallow fatty acid, are also suited. PEG 50 monostearate, PEG 100 monostearate, PEG 50 monooleate, PEG 100 monooleate, PEG 50 monolaurate and PEG 100 monolaurate are particularly preferred.

The use of C₁₂ to C₁₈ alkanols or C₁₂ to C₁₈ carboxylic acids, each comprising 10 to 30 units of ethylene oxide per mole, and mixtures of these substances, in particular Ceteth-10, Ceteth-12, Ceteth-20, Ceteth-30, Steareth-10, Steareth-12, Steareth-20, Steareth-21, Steareth-30, Ceteareth-10, Ceteareth-12, Ceteareth-20, Ceteareth-30, Laureth-12 and Beheneth-20, is particularly preferred. Preferred sorbitan monoesters, ethoxylated with an average of 20 to 100 moles ethylene oxide per mole, of linear saturated and unsaturated C₁₂ to C₃₀ carboxylic acids, which may be hydroxylated, are selected from Polysorbate-20, Polysorbate-40, Polysorbate-60 and Polysorbate-80.

Furthermore, preferably C₈ to C₂₂ alkyl monoglycosides and alkyl oligoglycosides are used. C₈ to C₂₂ alkyl monoglycosides and alkyl oligoglycosides are known, commercially available surfactants and emulsifiers. These are produced in particular by reacting glucose or oligosaccharides with primary alcohols having 8 to 22 carbon atoms. With respect to the glycoside group, it applies that both monoglycosides, in which a cyclic sugar group is glycosidically bound to the fatty alcohol, and oligomeric glycosides having a degree of oligomerization of up to approximately 8, preferably 1 to 2, are suited. The degree of oligomerization is a statistical mean value which is based on a distribution of homologs that is customary for such technical products. Products available under the trademark Plantacare® comprise a glucosidically bound C₈ to C₁₆ alkyl group on an oligoglucoside group, the mean degree of oligomerization is 1 to 2, in particular 1.2 to 1.4. Particularly preferred C₈ to C₂₂ alkyl monoglycosides and alkyl oligoglycosides are selected from the group consisting of octyl glucoside, decyl glucoside, lauryl glucoside, palmityl glucoside, isostearyl glucoside, stearyl glucoside, arachidyl glucoside, and behenyl glucoside, and mixtures thereof. The acyl glucamides derived from glucamine are also suited as non-ionic oil-in-water emulsifiers.

According to the invention, ethoxylated sterols, in particular ethoxylated soy sterols, also represent suitable oil-in-water emulsifiers. The degree of ethoxylation can be greater than 5, preferably at least 10, to have an HLB value greater than 7. Suitable commercial products are, for example, PEG-10 Soy Sterol, PEG-16 Soy Sterol, and PEG-25 Soy Sterol.

Furthermore, preferably partial esters of polyglycerols having 2 to 10 glycerol units and esterified with 1 to 4 saturated or unsaturated, linear or branched, optionally hydroxylated C₈ to C₃₀ fatty acid esters are used, provided they have an HLB value of greater than 7 to 20. Diglycerol monocaprylate, diglycerol monocaprate, diglycerol monolaurate, triglycerol monocaprylate, triglycerol monocaprate, triglycerol monolaurate, tetraglycerol monocaprylate, tetraglycerol monocaprate, tetraglycerol monolaurate, pentaglycerol monocaprylate, pentaglycerol monocaprate, pentaglycerol monolaurate, hexaglycerol monocaprylate, hexaglycerol monocaprate, hexaglycerol monolaurate, hexaglycerol monomyristate, hexaglycerol monostearate, decaglycerol monocaprylate, decaglycerol monocaprate, decaglycerol monolaurate, decaglycerol monomyristate, decaglycerol monoisostearate, decaglycerol monostearate, decaglycerol monooleate, decaglycerol monohydroxystearate, decaglycerol dicaprylate, decaglycerol dicaprate, decaglycerol dilaurate, decaglycerol dimyristate, decaglycerol diisostearate, decaglycerol distearate, decaglycerol dioleate, decaglycerol dihydroxystearate, decaglycerol tricaprylate, decaglycerol tricaprate, decaglycerol trilaurate, decaglycerol trimyristate, decaglycerol triisostearate, decaglycerol tristearate, decaglycerol trioleate, and decaglycerol dihydroxystearate.

Particularly preferred antiperspirant compositions according to the invention include at least one oil-in-water emulsifier having an HLB value of greater than 7 to 20 in a total amount of 0.5 to 5 wt. %, preferably 0.8 to 4 wt. %, particularly preferably 1.2 to 3 wt. %, and exceptionally preferably 1.5 to 2 wt. %, in each case based on the total composition.

Further particularly preferred antiperspirant compositions according to the invention include at least one non-ionic oil-in-water emulsifier having an HLB value in the range of 12 to 18 in a total amount of 0.5 to 5 wt. %, preferably 0.8 to 4 wt. %, particularly preferably 1.2 to 3 wt. %, and exceptionally preferably 1.5 to 2 wt. %, in each case based on the total composition.

Further particularly preferred antiperspirant compositions according to the invention include at least one non-ionic oil-in-water emulsifier having an HLB value in the range of 12 to 18, which is selected from linear saturated and unsaturated C₁₂ to C₂₄ alkanols that are etherified with 7 to 40 ethylene oxide units per molecule, in a total amount of 0.5 to 5 wt. %, preferably 0.8 to 4 wt. %, particularly preferably 1.2 to 3 wt. %, and exceptionally preferably 1.5 to 2 wt. %, in each case based on the total composition. It is particularly preferred when the aforementioned oil-in-water emulsifiers are selected from steareth, ceteth, myristeth, laureth, trideceth, arachideth and beheneth, each having 7 to 40 ethylene oxide units per molecule, in particular Steareth-10, Steareth-20, Steareth-21, Steareth-30, Steareth-40, Ceteth-10, Ceteth-20, Ceteth-21, Ceteth-30, Ceteth-40, Laureth-10, Laureth-20, Laureth-30, Trideceth-10, Trideceth-20 and Trideceth-30, and mixtures thereof.

Further preferred antiperspirant compositions according to the invention include at least one non-ionic oil-in-water emulsifier having an HLB value in the range of 12 to 18, which is selected from Steareth-10, Steareth-20, Steareth-21, Steareth-30, Steareth-40, Ceteth-10, Ceteth-20, Ceteth-21, Ceteth-30, Ceteth-40, Laureth-10, Laureth-20, Laureth-30, Trideceth-10, Trideceth-20 and Trideceth-30, and mixtures thereof, in a total amount of 0.5 to 5 wt. %, preferably 0.8 to 4 wt. %, particularly preferably 1.2 to 3 wt. %, and exceptionally preferably 1.5 to 2 wt. %, in each case based on the total composition.

Particularly preferred compositions according to the invention are characterized by comprising 0.1 to 10 wt. %, preferably 0.25 to 7.5 wt. %, more preferably 0.5 to 5 wt. %, particularly preferably 0.75 to 2.5 wt. %, and in particular 1 to 2 wt. % C₁₂ to C₁₈ alkanols and/or C₁₂ to C₁₈ carboxylic acids, each having 10 to 30 ethylene oxide units per molecule, preferably from the group consisting of Ceteth-12, Ceteth-20, Ceteth-30, Steareth-12, Steareth-20, Steareth-21, Steareth-30, Laureth-12, Beheneth-20, and the mixtures thereof.

Further preferred antiperspirant compositions according to the invention include at least one cosmetic oil and at least one oil-in-water emulsifier having an HLB value of greater than 7 to 20 and are present in the form of an oil-in-water emulsion. Within the meaning of the present application, the term ‘emulsion’ does not cover any microemulsions.

Particularly preferred antiperspirant compositions according to the invention are present in the form of an oil-in-water emulsion and include at least one oil-in-water emulsifier having an HLB value of greater than 7 to 20 in a total amount of 0.5 to 5 wt. %, preferably 0.8 to 4 wt. %, particularly preferably 1.2 to 3 wt. %, and exceptionally preferably 1.5 to 2 wt. %, in each case based on the total composition.

Further particularly preferred antiperspirant compositions according to the invention are present in the form of an oil-in-water emulsion and include at least one non-ionic oil-in-water emulsifier having an HLB value in the range of 12 to 18 in a total amount of 0.5 to 5 wt. %, preferably 0.8 to 4 wt. %, particularly preferably 1.2 to 3 wt. %, and exceptionally preferably 1.5 to 2 wt. %, in each case based on the total composition.

Further particularly preferred antiperspirant compositions according to the invention are present in the form of an oil-in-water emulsion and include at least one non-ionic oil-in-water emulsifier having an HLB value in the range of 12 to 18, which is selected from linear saturated and unsaturated C₁₂ to C₂₄ alkanols that are etherified with 7 to 40 ethylene oxide units per molecule, in a total amount of 0.5 to 5 wt. %, preferably 0.8 to 4 wt. %, particularly preferably 1.2 to 3 wt. %, and exceptionally preferably 1.5 to 2 wt. %, in each case based on the total composition. It is particularly preferred when the aforementioned oil-in-water emulsifiers are selected from steareth, ceteth, myristeth, laureth, trideceth, arachideth and beheneth, each having 7 to 40 ethylene oxide units per molecule, in particular Steareth-10, Steareth-20, Steareth-21, Steareth30, Steareth-40, Ceteth-10, Ceteth-20, Ceteth-21, Ceteth-30, Ceteth-40, Laureth-10, Laureth-20, Laureth-30, Trideceth-10, Trideceth-20 and Trideceth-30, and mixtures thereof.

Further preferred antiperspirant compositions according to the invention are present in the form of an oil-in-water emulsion and include at least one non-ionic oil-in-water emulsifier having an HLB value in the range of 12 to 18, which is selected from Steareth-10, Steareth-20, Steareth-21, Steareth30, Steareth-40, Ceteth-10, Ceteth-20, Ceteth-21, Ceteth-30, Ceteth-40, Laureth-10, Laureth-20, Laureth-30, Trideceth-10, Trideceth-20 and Trideceth-30, and mixtures thereof, in a total amount of 0.5 to 5 wt. %, preferably 0.8 to 4 wt. %, particularly preferably 1.2 to 3 wt. %, and exceptionally preferably 1.5 to 2 wt. %, in each case based on the total composition. Water-in-oil emulsifiers

Further preferred antiperspirant compositions according to the invention include at least one water-in-oil emulsifier, preferably at least one non-ionic water-in-oil emulsifier, in each case having an HLB value of greater than 1.0 and smaller than/equal to 7.0, preferably in the range of 3 to 6. Several of these water-in-oil emulsifiers are listed in Kirk-Othmer, “Encyclopedia of Chemical Technology”, 3rd edition, 1979, Volume 8, page 913, for example. For ethoxylated adducts, the HLB value can also be calculated, as was already mentioned.

Preferred water-in-oil emulsifiers are:
linear or branched, saturated or unsaturated C₁₂ to C₃₀ alkanols, each being etherified with 1 to 4 ethylene oxide units per molecule, which are exceptionally preferably selected from steareth, ceteth, myristeth, laureth, trideceth, arachideth and beheneth, each having 1 to 4 ethylene oxide units per molecule, in particular Steareth-2, Steareth-3, Steareth-4, Ceteth-2, Ceteth-3, Ceteth-4, Myristeth-2, Myristeth-3, Myristeth-4, Laureth-2, Laureth-3, Laureth-4, Trideceth-2, Trideceth-3 and Trideceth-4, and mixtures thereof;
linear saturated alkanols comprising 12 to 30 carbon atoms, in particular comprising 16 to 22 carbon atoms, in particular cetyl alcohol, stearyl alcohol, arachidyl alcohol, behenyl alcohol, and lanolin alcohol, or mixtures of these alcohols, as they are obtainable from the technical hydrogenation of vegetable and animal fatty acids;
esters, and in particular partial esters, made of a polyol having 2 to 6 carbon atoms, and linear saturated and unsaturated fatty acids having 12 to 30, in particular 14 to 22, carbon atoms, which can be hydroxylated. Such esters or partial esters are, for example, the monoesters and diesters of glycerol or ethylene glycol, or the monoesters of propylene glycol having linear saturated and unsaturated C₁₂ to C₃₀ carboxylic acids, which can be hydroxylated, in particular those comprising palmitic and stearic acid, the sorbitan monoesters, diesters or triesters of linear saturated and unsaturated C₁₂ to C₃₀ carboxylic acids, which can be hydroxylated, in particular those of myristic acid, palmitic acid, stearic acid, or mixtures of these fatty acids, the pentaerythrityl monoesters, diesters, triesters and tetraesters, and the methyl glucose monoesters and diesters of linear, saturated and unsaturated C₁₂ to C₃₀ carboxylic acids, which can be hydroxylated, of which the monoesters, diesters, triesters and tetraesters of pentaerythritol comprising linear saturated fatty acids having 12 to 30, in particular 14 to 22, carbon atoms, which can be hydroxylated, and mixtures thereof, are particularly preferred as stabilizers and/or water binders. The monoesters and diesters are particularly preferred according to the invention. Preferred C₁₂ to C₃₀ fatty acid groups according to the invention are selected from lauric acid, myristic acid, palmitic acid, stearic acid, arachinic acid and behenic acid groups, the stearic acid group being particularly preferred. Particularly preferred non-ionic water-in-oil emulsifiers according to the invention, having an HLB value of greater than 1.0 and smaller than/equal to 7.0, are selected from glyceryl monostearate, glyceryl distearate, glyceryl monopalmitate, glyceryl dipalmitate, and mixtures thereof;
sterols, which is to say steroids, which carry a hydroxyl group at the C3 atom of the steroid skeleton and are isolated both from animal tissue (zoosterols, such as cholesterol, lanosterol), from plants (phytosterols, such as ergosterol, stigmasterol, sitosterol) and from fungi and yeasts (mycosterols) and which may be low-ethoxylated (1 to 5 EO);
alkanols and carboxylic acids, each having 8 to 24 C atoms, in particular having 16 to 22 C atoms, in the alkyl groups and 1 to 4 ethylene oxide units per molecule, which have an HLB value of greater than 1.0 and/or smaller than/equal to 7.0;
glycerol monoethers of saturated and/or unsaturated, branched and/or unbranched alcohols having a chain length of 8 to 30, in particular 12 to 18 carbon atoms;
partial esters of polyglycerols having n=2 to 10 glycerol units and esterified with 1 to 5 saturated or unsaturated, linear or branched, optionally hydroxylated C₈ to C₃₀ fatty acid esters, provided they have an HLB value of greater than 1.0 to smaller than/equal to 7;
and mixtures of the aforementioned substances.

The at least one water-in-oil emulsifier having an HLB value greater than 1.0 and smaller than/equal to 7.0, preferably in the range of 3 to 6, is particularly preferably selected from linear or branched, saturated or unsaturated C₁₂ to C₃₀ alkanols, each being etherified with 1 to 4 ethylene oxide units per molecule, which are exceptionally preferably selected from steareth, ceteth, myristeth, laureth, trideceth, arachideth and beheneth, each having 1 to 4 ethylene oxide units per molecule, in particular Steareth-2, Steareth-3, Steareth-4, Ceteth-2, Ceteth-3, Ceteth-4, Myristeth-2, Myristeth-3, Myristeth-4, Laureth-2, Laureth-3, Laureth-4, Trideceth-2, Trideceth-3 and Trideceth-4, and mixtures thereof. It may be preferred according to the invention to use only a single water-in-oil emulsifier. In another preferred embodiment, the compositions according to the invention include mixtures, in particular technical mixtures, of at least two water-in-oil emulsifiers. Preferred antiperspirant compositions according to the invention include at least one water-in-oil emulsifier having an HLB value greater than 1.0 and smaller than/equal to 7.0, preferably in the range of 3 to 6, in a total amount of 1.8 to 3 wt. %, preferably 2 to 2.8 wt. %, and particularly preferably 2.4 to 2.6 wt. %, in each case based on the total weight of the composition according to the invention.

Further preferred antiperspirant compositions according to the invention include at least one non-ionic water-in-oil emulsifier having an HLB value in the range of 3 to 6, which is selected from Steareth-2, Steareth-3, Steareth-4, Ceteth-2, Ceteth-3, Ceteth-4, Myristeth-2, Myristeth-3, Myristeth-4, Laureth-2, Laureth-3, Laureth-4, Trideceth-2, Trideceth-3 and Trideceth-4, and mixtures thereof, in a total amount of 1.8 to 3 wt. %, preferably 2 to 2.8 wt. %, and particularly preferably 2.4 to 2.6 wt. %, in each case based on the total weight of the composition according to the invention.

Further preferred antiperspirant compositions according to the invention are present in the form of an oil-in-water emulsion and include at least one water-in-oil emulsifier having an HLB value greater than 1.0 and smaller than/equal to 7.0, preferably in the range of 3 to 6, in a total amount of 1.8 to 3 wt. %, preferably 2 to 2.8 wt. %, and particularly preferably 2.4 to 2.6 wt. %, in each case based on the total weight of the composition according to the invention.

Further preferred antiperspirant compositions according to the invention are present in the form of an oil-in-water emulsion and include at least one non-ionic water-in-oil emulsifier having an HLB value in the range of 3 to 6, which is selected from Steareth-2, Steareth-3, Steareth-4, Ceteth-2, Ceteth-3, Ceteth-4, Myristeth-2, Myristeth-3, Myristeth-4, Laureth-2, Laureth-3, Laureth-4, Trideceth-2, Trideceth3 and Trideceth-4, and mixtures thereof, in a total amount of 1.8 to 3 wt. %, preferably 2 to 2.8 wt. %, and particularly preferably 2.4 to 2.6 wt. %, in each case based on the total weight of the composition according to the invention.

Preferred antiperspirant compositions according to the invention are present in the form of an oil-in-water emulsion and include at least one cosmetic oil, preferably in a total amount of 0.1 to 15 wt. %, particularly preferably 0.3 to 10 wt. %, exceptionally preferably 0.5 to 6 wt. %, in each case based on the weight of the total antiperspirant composition according to the invention.

A distinction is made between volatile and non-volatile oils in terms of the cosmetic oils. Non-volatile oils are understood to mean oils that have a vapor pressure of less than 2.66 Pa (0.02 mm Hg) at 20° C. and an ambient pressure of 1013 hPa. Volatile oils are understood to mean oils that have a vapor pressure of 2.66 Pa to 40000 Pa (0.02 to 300 mm Hg), preferably 13 to 12000 Pa (0.1 to 90 mm Hg), particularly preferably 15 to 3000 Pa, exceptionally preferably 30 to 500 Pa, at 20° C. and an ambient pressure of 1013 hPa.

Particularly preferred non-volatile non-silicone oils according to the invention are selected from the addition products of at least 6 ethylene oxide units and/or propylene oxide units to monohydric or polyhydric C₃₋₂₂ alkanols, such as butanol, butanediol, myristyl alcohol and stearyl alcohol, such as PPG-13 butyl ether, PPG-14 butyl ether, PPG-9 butyl ether, PPG-10 butanediol, PPG-15 stearyl ether, and mixtures thereof.

Particularly preferred compositions according to the invention include at least one cosmetic oil selected from PPG-13 butyl ether, PPG-14 butyl ether, PPG-9 butyl ether, PPG-10 butanediol, PPG-15 stearyl ether, and mixtures thereof, in a total amount of 0.1 to 15 wt. %, particularly preferably 0.3 to 10 wt. %, exceptionally preferably 0.5 to 6 wt. %, based on the weight of the total antiperspirant composition according to the invention. Exceptionally preferred compositions according to the invention include 0.1 to 15 wt. %, particularly preferably 0.3 to 10 wt. %, exceptionally preferably 0.5 to 6 wt. %, PPG-15 stearyl ether, in each case based on the weight of the total antiperspirant composition according to the invention.

Further particularly preferred non-volatile non-silicone oils according to the invention are esters of the linear or branched, saturated or unsaturated fatty alcohols comprising 2 to 30 carbon atoms having linear or branched, saturated or unsaturated fatty acids having 2 to 30 carbon atoms, which can be hydroxylated. Esters of the linear or branched saturated fatty alcohols comprising 2 to 5 carbon atoms having linear or branched, saturated or unsaturated fatty acids having 10 to 18 carbon atoms, which can be hydroxylated, are preferred. Preferred examples in this regard are isopropyl palmitate, isopropyl stearate, isopropyl myristate, 2-hexyldecyl stearate, 2-hexyldecyl laurate, isononyl isononanoate, 2-ethylhexyl palmitate, and 2-ethylhexyl stearate. Likewise preferred are isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid-2-butyl octanoate, diisotridecyl acetate, n-hexyl laurate, n-decyl oleate, oleyl oleate, oleyl erucate, erucyl oleate, C₁₂₋₁₅ alkyl lactate, and di-C₁₂₋₁₃ alkyl malate, and the benzoic acid esters of linear or branched C₈₋₂₂ alkanols. Particularly preferred are benzoic acid-C₁₂₋₁₅-alkyl esters, for example available as the commercial product Finsolv® TN (C₁₂₋₁₅ alkyl benzoate), and benzoic acid isostearyl esters, for example available as Finsolv® SB, 2-ethylhexyl benzoate, for example available as Finsolv® EB, and benzoic acid-2-octyldodecyl ester, for example available as Finsolv® BOD. A further particularly preferred ester oil is triethyl citrate.

Further preferred non-volatile non-silicone oils according to the invention are selected from branched saturated or unsaturated fatty alcohols having 6 to 30 carbon atoms. These alcohols are frequently also referred to as Guerbet alcohols since they can be obtained by way of the Guerbet reaction. Preferred alcohol oils are 2-hexyldecanol, 2-octyldodecanol and 2-ethylhexyl alcohol. Isostearyl alcohol is likewise preferred. Further preferred non-volatile oils are selected from mixtures of Guerbet alcohols and Guerbet alcohol esters, such as 2-hexyldecanol and 2-hexyldecyl laurate.

The expression “triglyceride” used hereafter shall be understood to mean “triesters of glycerol.” Further preferred non-volatile oils according to the invention are selected from the triglycerides of linear or branched, saturated or unsaturated, optionally hydroxylated C₈ to C₃₀ fatty acids, provided these are liquid under normal conditions. The use of natural oils can be particularly suitable, such as soy bean oil, cottonseed oil, sunflower oil, palm oil, palm kernel oil, linseed oil, almond oil, castor oil, corn oil, rapeseed oil, olive oil, sesame oil, safflower oil, wheat germ oil, peach kernel oil, and the liquid components of coconut oil, and the like. Synthetic triglyceride oils are particularly preferred, in particular capric/caprylic triglycerides, such as the commercial products Myritol® 318 or Myritol® 331 (BASF/Cognis) comprising unbranched fatty acid esters and glyceryl triisostearyl and glyceryl tri(2-ethylhexanoate) comprising branched fatty acid esters. Such triglyceride oils preferably account for a proportion of less than 50 wt. % of the total weight of all cosmetic oils in the composition according to the invention.

Further particularly preferred non-volatile non-silicone oils according to the invention are selected from the dicarboxylic acid esters of linear or branched C₂-C₁₀ alkanols, in particular diisopropyl adipate, di-n-butyl adipate, di-(2-ethylhexyl) adipate, dioctyl adipate, diethyl-/di-n-butyl/dioctyl sebacate, diisopropyl sebacate, dioctyl malate, dioctyl maleate, dicaprylyl maleate, diisooctyl succinate, di-2-ethylhexyl succinate, and di-(2-hexyldecyl) succinate.

Further particularly preferred non-volatile non-silicone oils according to the invention are selected from the addition products of 1 to 5 ethylene oxide units to monohydric or polyhydric C₈₋₂₂ alkanols, such as octanol, decanol, decandiol, lauryl alcohol, myristyl alcohol, and stearyl alcohol, preferably made of PPG-2 myristyl ether end PPG-3 myristyl ether.

Further particularly preferred non-volatile non-silicone oils according to the invention are selected from the symmetrical, asymmetrical or cyclic esters of carbonic acid comprising C₆ to C₂₀ alcohols, such as di-n-caprylyl carbonate (Cetiol® CC) or di-(2-ethylhexyl) carbonate (Tegosoft DEC). Esters of carbonic acid comprising C₁ to C₅ alcohols, such as glycerol carbonate or propylene carbonate, in contrast, are not compounds suitable as cosmetic oils.

Further oils that may be preferred according to the invention are selected from the esters of dimers of unsaturated C₁₂ to C₂₂ fatty acids (dimer fatty acids) comprising monohydric linear, branched or cyclic C₂ to C₁₈ alkanols or polyhydric linear or branched C₂ to C₆ alkanols. The total weight of dimeric fatty acid esters is particularly preferably 0.5 to 10 wt. %, preferably 1 to 5 wt. %, in each case based on the total composition.

Volatile cosmetic oils are usually selected from among the cyclic silicone oils having the INCI name Cyclomethicone. The INCI name Cyclomethicone shall be understood to mean in particular cyclotrisiloxane (hexamethyl cyclotrisiloxane), cyclotetrasiloxane (octamethyl cyclotetrasiloxane), cyclopentasiloxane (decamethyl cyclopentasiloxane) and cyclohexasiloxane (dodecamethyl cyclohexasiloxane). These oils have a vapor pressure of approximately 13 to 15 Pa at 20° C.

Cyclomethicones are known in the prior art as oils well-suited for cosmetic products, in particular for antiperspirant and deodorant products. However, due to the persistence of the same in the environment, it may be preferred according to the invention to dispense with the use of cyclomethicones. In a specifically preferred embodiment, the compositions according to the invention include 0 to less than 1 wt. % cyclomethicone, based on the weight of the composition.

A preferred cyclomethicone substitute is a mixture of C₁₃ to C₁₆ isoparaffins, C₁₂ to C₁₄ isoparaffins, and C₁₃ to C₁₅ alkanes, the viscosity of which at 25° C. is in a range of 2 to 6 mPas and which have a vapor pressure at 20° C. in the range of 100 to 150 Pa. Such a mixture is available, for example, under the designation SiClone SR-5 from Presperse Inc.

Further preferred volatile silicone oils are selected from volatile linear silicone oils, in particular volatile linear silicone oils having 2 to 10 siloxane units, such as hexamethyldisiloxane (L2), octamethyltrisiloxane (L3), decamethyltetrasiloxane (L4), as they are present, for example, in the commercial products DC 2-1184, Dow Corning® 200 (0.65 cSt) and Dow Corning 200 (1.5 cSt) from Down Corning, and low molecular weight phenyl trimethicone having a vapor pressure at 20° C. of approximately 2000 Pa, as it is available from GE Bayer Silicones/Momentive, for example, under the name Baysilone Fluid PD 5.

Due to the drier skin sensation and faster active ingredient release, preferred antiperspirant compositions according to the invention include at least one volatile non-silicone oil. Preferred volatile non-silicone oils are selected from C₆ to C₁₆ isoparaffins, in particular from isononane, isodecane, isoundecane, isododecane, isotridecane, isotetradecane, isopentadecane, and isohexadecane, and mixtures thereof. Preferred are C₁₀ to C₁₃ isoparaffin mixtures, in particular those having a vapor pressure at 20° C. of 10 to 400 Pa, preferably 13 to 100 Pa.

Further preferred antiperspirant compositions according to the invention in the form of oil-in-water emulsions are characterized in that the at least one propylene glycol monoester of branched saturated C₆ to C₃₀ alkane carboxylic acids is selected from propylene glycol monoisostearate, propylene glycol monoisopalmitate, propylene glycol monoisobehenate, propylene glycol monoisoarachinate, propylene glycol monoisomyristate, propylene glycol monoisocaprate, propylene glycol monoisocaprinate, and propylene glycol monoisocaprylate, and mixtures thereof. Further preferred antiperspirant compositions according to the invention in the form of oil-in-water emulsions are characterized in that the at least one branched, saturated C₁₀ to C₃₀ alkanol is selected from isostearyl alcohol, isocetyl alcohol, isomyristyl alcohol, isotridecyl alcohol, isoarachidyl alcohol, isobehenyl alcohol, isocapryl alcohol, isocaprinyl alcohol, isocaprylyl alcohol, and mixtures thereof. Further preferred antiperspirant compositions according to the invention in the form of oil-in-water emulsions are characterized in that at least one non-ionic emulsifier having an HLB value in the range of 3 to 6 and at least one non-ionic emulsifier having an HLB value in the range of 12 to 18 are present.

Further preferred antiperspirant compositions according to the invention in the form of oil-in-water emulsions are characterized in that at least one non-ionic emulsifier having an HLB value in the range of 3 to 6 is present in a total quantity of 1.8 to 3 wt., and at least one non-ionic emulsifier having an HLB value in the range of 12 to 18 is present in a total amount of 1 to 2 wt., wherein the quantity information in each case is based on the total weight of the composition according to the invention.

Further preferred antiperspirant compositions according to the invention in the form of oil-in-water emulsions are characterized in that Steareth-2 is present as a non-ionic emulsifier having an HLB value in the range of 3 to 6, and Steareth-21 is present at the same time as a non-ionic emulsifier having an HLB value in the range of 12 to 18.

Further preferred antiperspirant compositions according to the invention in the form of oil-in-water emulsions are characterized in that Steareth-2, Steareth-21 and PPG-15 stearyl ether are present.

Further preferred antiperspirant compositions according to the invention in total include a maximum of 3 wt. %, preferably a maximum of 1 wt. %, and particularly preferably 0 wt. % of monohydric C₁ to C₃ alkanols, such as ethanol or isopropanol, in each case based on the total weight of the composition according to the invention.

In addition to the ingredients mentioned above, the compositions according to the invention can include further additives and auxiliary substances, which improve the shelf life of the same, for example, such as preservatives, for example phenoxyethanol, methylparaben or propylparaben, antioxidants, such as tetradibutyl pentaerythrityl hydroxyhydrocinnamate, Lipochroman-6, tocopherol, tocopheryl acetate or ascorbic acid and the derivatives thereof, vitamins and the derivatives thereof, such as tocopherol, tocopheryl acetate, ascorbic acid, panthenol or pantolactone, perfumes, essential oils, menthol and menthol derivatives exhibiting skin-cooling action, care substances having skin-soothing action, such as bisabolol and allantoin, active ingredients that slow hair growth, such as eflornithine or glycyrrhizin and the derivatives thereof, moisturizers and humectants such as 1,2-propylene glycol, glycerol, 2-methyl-1,3-propanediol, 1,2-butylene glycol, 1,3-butylene glycol, 1,4-butylene glycol, pentylene glycols such as 1,2-pentanediol and 1,5-pentanediol, hexanediols such as 1,2-hexanediol and 1,6-hexanediol, hexanetriols such as 1,2,6-hexanetriol, 1,2-octanediol, 1,8-octanediol, dipropylene glycol, tripropylene glycol, diglycerol, triglycerol, erythritol, sorbitol, cis-1,4-dimethylol cyclohexane, trans-1,4-dimethylol cyclohexane, arbitrary isomer mixtures of cis- and trans-1,4-dimethylol cyclohexane, urea, N,N′-bis-(2-hydroxyethyl) urea, sodium pyrrolidone carboxylate, plant extracts such as aloe vera extract, natural fats and oils such as jojoba oil, evening primrose oil or linseed oil, saturated and unsaturated fatty acids such as stearic acid, oleic acid, linoleic acid, linolenic acid or gamma-linolenic acid, squalane, squalene, active deodorant ingredients such as silver salts, colloidal silver, zeolites, 2-benzylheptane-1-ol, anise alcohol, mixtures of 2-benzylheptane-1-ol and phenoxyethanol, 3-(2-ethylhexyloxy)-1,2-propanediol or tropolone, and mixtures of these substances.

The present invention further relates to the use of at least one active ingredient, selected from:
i) taurine,
ii) glycine,
iii) ectoine,
   in combination with at least one active ingredient, selected from:
iv) ethylenediaminetetraacetate,
v) ethylenediaminetetraacetic acid and/or the salts thereof,
vi) ethylenediaminedisuccinic acid and/or the salts thereof,
vii) citric acid and/or the salts thereof,
viii) gluconic acid and/or the salts thereof,
ix) zeolite A,
x) sodium tripolyphosphate,
xi) sodium hexametaphosphate,
   in a cosmetically or dermatologically tolerable carrier, comprising at least one active antiperspirant ingredient, to improve the skin tolerance and/or the nourishing action of the composition and/or to reduce or prevent stinging of the skin and/or itching caused by the contact of the skin with the composition.

What was said with respect to the agents according to the invention applies, mutatis mutandis, with respect to preferred embodiments of the use according to the invention.

The following exemplary embodiments are intended to illustrate the subject matter of the present invention, without thereby limiting the invention to these examples.

### Examples

All information in wt. %

| | E1 | E2 | E3 | E4 | E5 | E6 | E7 | E8 | E9 | E10 | E11 | E12 | E13 | E14 | E15 | e16 |
|---|---|---|---|---|---|---|---|---|---|---|---|---|---|---|---|---|
| Aluminum | 20.0 | 20.0 | 20.0 | 20.0 | 20.0 | 20.0 | 20.0 | 20.0 | 20.0 | 20.0 | 20.0 | 20.0 | 20.0 | 20.0 | 20.0 | 20.0 |
| chlorohydrate | | | | | | | | | | | | | | | | |
| Steareth-21 | 1.5 | 1.5 | 1.5 | 1.5 | 1.5 | 1.5 | 1.5 | 1.5 | 1.5 | 1.5 | 1.5 | 1.5 | 1.5 | 1.5 | 1.5 | 1.5 |
| Steareth-2 | 2.4 | 2.4 | 2.4 | 2.4 | 2.4 | 2.4 | 2.4 | 2.4 | 2.4 | 2.4 | 2.4 | 2.4 | 2.4 | 2.4 | 2.4 | 2.4 |
| PPG-15 | 0.5 | 0.5 | 0.5 | 0.5 | 0.5 | 0.5 | 0.5 | 0.5 | 0.5 | 0.5 | 0.5 | 0.5 | 0.5 | 0.5 | 0.5 | 0.5 |
| stearyl ether | | | | | | | | | | | | | | | | |
| EDTA | 0.1 | — | 0.1 | — | 0.1 | — | 0.1 | — | 0.05 | — | 0.05 | — | 0.05 | 0.05 | 0.05 | 0.05 |
| EDDS | — | 0.1 | — | 0.1 | — | 0.1 | — | 0.1 | — | 0.05 | — | 0.05 | 0.05 | 0.05 | 0.05 | 0.05 |
| Taurine | 1.0 | 1.0 | — | — | 0.5 | 0.5 | — | — | 1.0 | 1.0 | — | — | 1.0 | — | 0.5 | 0.5 |
| Ectoine | — | — | 1.0 | 1.0 | — | — | 0.5 | 0.5 | — | — | 1.0 | 1.0 | — | 1.0 | 0.5 | 0.5 |

| | |
|---|---|
| Water | to make up to 100 |

The agent E1 according to the invention was compared to the comparison agents V1 and V2, which were free of EDTA and/or taurine:

| | E1 | V1 | V2 |
|---|---|---|---|
| Aluminum chlorohydrate | 20.0 | 20.0 | 20.0 |
| Steareth-21 | 1.5 | 1.5 | 1.5 |
| Steareth-2 | 2.4 | 2.4 | 2.4 |
| PPG-15 stearyl ether | 0.5 | 0.5 | 0.5 |
| EDTA | 0.1 | — | 0.1 |
| Taurine | 1.0 | 1.0 | — |

| | |
|---|---|
| Water | to make up to 100 |

The agents were provided to test persons with sensitive skin for three weeks for normal use, wherein a different agent was applied each week. The agents were evaluated according to the following grading pattern:

1 no itching effect perceptible
2 slight itching effect perceptible
3 itching effect perceptible
4 strong itching effect
5 very strong itching effect
6 intolerable itching effect

The mean values from the grading of the 20 test persons is shown in the following table:

| | E1 | V1 | V2 |
|---|---|---|---|
| Average grade | 1.4 | 2.2 | 4.2 |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic or dermatological antiperspirant composition, comprising, based on the weight thereof,
a) at least one active antiperspirant ingredient,
b) 0 to 5 wt. % taurine,
c) 0 to 5 wt. % glycine,
d) 0 to 5 wt. % ectoine,
e) 0 to 2 wt. % ethylenediaminetetraacetic acid and/or the salts thereof,
f) 0 to 2 wt. % ethylenediaminedisuccinic acid and/or the salts thereof,
g) 0 to 2 wt. % citric acid and/or the salts thereof,
h) 0 to 2 wt. % gluconic acid and/or the salts thereof,
i) 0 to 2 wt. % zeolite A,
j) 0 to 2 wt. % sodium tripolyphosphate,
k) 0 to 2 wt. % sodium hexametaphosphate,
with the proviso that
the total amount of ingredients b), c) and d) is 0.1 to 5 wt. %, and
the total amount of ingredients e), f), g), h), i), j) and k) is 0.01 to 2 wt. %.

2. The compositions according to claim 1, wherein the at least one activated antiperspirant aluminum or aluminum-zirconium salt comprises 3 to 25 wt. % of the composition.

3. The composition according to claim 1, characterized by comprising at least one antiperspirant aluminum salt in a total amount of 2 to 40 wt. %, preferably 8 to 35 wt. %, particularly preferably 10 to 28 wt. %, and exceptionally preferably 12 to 20 wt. %, the information in % by weight being based on the total weight of the constitutional water-free and ligand-free active substance (USP) in the composition.

4. The composition according to claim 1, wherein the taurine comprises 0.2 to 4 wt. % of the composition.

5. The composition according to claim 1, wherein the ectoine comprises 0.1 to 5 wt. %, preferably 0.2 to 4 wt. % of the composition.

6. The composition according to claim 1, wherein the ethylenediaminetetraacetic acid and/or salts thereof comprises 0.01 to 2 wt. % of the composition.

7. The composition according to claim 1, wherein the ethylenediaminetetraacetic acid and/or salts thereof comprises 0.02 to 1 wt. % of the composition.

8. The composition according to claim 1, wherein ethylenediaminedisuccinic acid and/or the salts thereof comprises 0.01 to 2 wt. % of the composition.

9. The composition according to claim 1, wherein ethylenediaminedisuccinic acid and/or the salts thereof comprises 0.02 to 1 wt. % of the composition.

10. The composition according to claim 1, wherein the composition includes 0.2 to 4 wt. %, taurine, and 0.01 to 2 wt. % ethylenediaminetetraacetic acid and/or the salts thereof.

11. The composition according to claim 10, wherein the weight ratio of taurine to ethylenediaminetetraacetic acid and/or the salts thereof is 2:11 to 100:1.

12. The composition according to claim 1, wherein the composition includes ectoine and 0.01 to 2 wt. % sodium tripolyphosphate.

13. The composition according to claim 1, wherein the composition includes 0.1 to 5 wt. % actoine and 0.01 to 2 wt % sodium hexametaphosphate.

14. The composition according to claim 1, wherein the composition includes 0.1 to 5 wt. % taurine and 0.01 to 2 wt. % zeolite A.

15. The composition according to claim 1, wherein zeolite A comprises 0.1 to 5 wt. % of the composition.

16. The composition according to claim 1, wherein zeolite A comprises 0.4 to 2 wt. % of the composition.

17. The composition according to claim 1, wherein the composition comprises 10 to 80 wt. % water based on the total weight of the composition.

18. The composition according to claim 1, wherein the composition further comprises 0.1 to 10 wt. % C₁₂ to C₁₈ alkanols and/or C₁₂ to C₁₈ carboxylic acids, each having 10 to 30 ethylene oxide units per molecule, based on the total weight of the composition.

19. The composition according to claim 18, wherein the C₁₂ to C₁₈ alkanols and/or C₁₂ to C₁₈ carboxylic acids, each having 10 to 30 ethylene oxide units per molecule, is selected from group consisting of: Ceteth-12, Ceteth-20, Ceteth-30, Steareth-12, Steareth-20, Steareth-21, Steareth-30, Laureth-12, Beheneth-20, and the mixtures thereof.
